(19) United States
(12) Patent Application Publication
(10) Pub. No.: US 2013/0046323 A1
(43) Pub. Date: Feb. 21, 2013

(54) SURGICAL SCISSORS AND METHOD OF MAKING AND USING THE SAME

(71) Applicant: David WHITAKER, San Clemente, CA (US)

(72) Inventor: David WHITAKER, San Clemente, CA (US)

(73) Assignee: MEDIX INC., Ponte Vedra Beach, FL (US)

(21) Appl. No.: 13/586,382

(22) Filed: Aug. 15, 2012

Related U.S. Application Data
(60) Provisional application No. 61/524,084, filed on Aug. 16, 2011.

(51) Int. Cl. A61B 17/3201

(52) U.S. Cl. 606/174

(57) ABSTRACT

Scissors including a first blade member, a second blade member movably connected to the first blade member, and at least one of indicia arranged on at least one of the first and second blade members, indicia formed in at least one of the first and second blade members, and an optional reflex hammer can be arranged on at least one of the first and second blade members.

### CROSS-REFERENCE TO RELATED APPLICATIONS

The instant application is a US non-provisional Application that is based on and claims the benefit of U.S. provisional application No. 61/524,084, filed Aug. 16, 2011, the disclosure of which is hereby expressly incorporated by reference herein in its entirety.

### BACKGROUND OF THE INVENTION

1. Field of the Invention

This invention relates generally to medical, bandage and/or surgical scissors or shears. The invention also relates to a method of using the scissors, as well as a method of making the scissors.

2. Discussion of Background Information

Medical scissors are known from, for example, the following link http://www.alibaba.com/product-gs/257308823/Bandages_cissors_Surgical_scissors_medical_scissor/showimage.html. The disclosure of this document is expressly incorporated by reference herein in its entirety. However, such scissors lack indicia on the blade which can indicate length values and/or angle valves. Nor do they utilize or integrate a reflex hammer.

Also known are scissors with a hammer feature as evidenced by the following documents: U.S. Pat. No. 4,485,507 to KANTWERK and U.S. Pat. No. 4,658,456 to TSAI. The disclosure of each of these documents is expressly incorporated by reference herein in their entireties.

Other scissors are known as evidenced by the following documents: US 2008/0045990 to ZELINKO, US 2008/0289187 to FISHER, US 2009/0271987 to FISHER, U.S. Pat. No. 7,166,119 to OLSEN, U.S. Pat. No. 5,014,433 to TEPIC, U.S. Pat. No. 4,819,636 to GERICH et al. The disclosure of each of these documents is expressly incorporated by reference herein in their entireties.

### SUMMARY OF THE INVENTION

The invention aims to improve devices of the type described above by making scissors having one, more than one, or preferably most or all of the features shown in the drawings which are not shown in the above-noted documents.

According to one non-limiting aspect of the invention, there is provided scissors having one or more features shown in the drawings (which are approximately to scale), described herein and/or recited in the claims. The overall length of at least one embodiment shown in the drawings is approximately 8 inches (approximately 20 cm). The scissors can also be sized between up to about 50 percent smaller in scale from that shown to up to about 30 percent larger in scale, as well as any whole percentage value in between.

In embodiments, the blade members are made of stainless-steel and the finger grips are made of synthetic resin.

In embodiments, the blade members are made of surgical grade metal and the finger grips are made of medical grade plastic or synthetic resin.

In embodiments, the blade members are made of surgical grade stainless-steel sheet-metal and the finger grips are made of medical grade plastic or synthetic resin.

In embodiments, the blade members are made of surgical grade steel and the finger grips are over-molded onto the blade members.

In embodiments, the blade members are metal colored and the finger grips are of a different color or shade of color.

In embodiments, the blade members are pivotally connected to one another via a rivet or pin.

In embodiments, at least one of the blade members has indicia on the portions of the blade members that cut against one another. In embodiments, the indicia includes angle values or values in degrees. In embodiments, the indicia includes length values or values in inches or centimeters. In embodiments, the indicia includes both angle values or values in degrees and length values or values in inches or centimeters.

In embodiments, at least one of the blade members has a finger grip which is configured for use as a reflex hammer.

In embodiments, at least one of the blade members has a finger grip sized for a thumb which is configured and shaped for use as a reflex hammer.

In embodiments, one of the blade members has a finger grip sized for a fore finger and middle finger.

In embodiments, the two blade members have finger grips whose openings are sized to ergonomically accommodate a user's fingers and thumb.

In embodiments, the scissors can include any one, more than one, or all of the features disclosed in pending U.S. Design application Ser. No. 29/367,843 filed Aug. 13, 2010 (attorney docket D38765), the disclosure of which is hereby expressly incorporated by reference in its entirety.

The invention also provides for a method of using the scissors described above, wherein the method comprises cutting tissue and/or material during a medical environment and/or during a medical procedure. The invention also provides for a method of making the scissors as shown and/or described herein.

According to one non-limiting aspect of the invention there is provided scissors having one or more features shown in the above noted “http” link, as well as one or more features shown in the drawings of the instant application such as the indicia and/or the reflex hammer shaped finger/thumb grip.

According to one non-limiting aspect of the invention there is provided a scissors comprising a first blade member, a second blade member movably connected to the first blade member, and at least one of indicia arranged on at least one of the first and second blade members and indicia formed in at least one of the first and second blade members.

In embodiments, the scissors may further comprise a reflex hammer arranged on a handle coupled to at least one of the first and second blade members.

In embodiments, the scissors are one of surgical scissors, medical scissors and bandage scissors.

In embodiments, the indicia is arranged on a first blade member and a reflex hammer is coupled to the first blade member.

In embodiments, the indicia comprises at least one of length indicia and angle indicia.

In embodiments, the indicia comprises length indicia and angle indicia.

In embodiments, the indicia is arranged on the first blade member adjacent a cutting portion of the first blade member and a lever portion of the first blade member comprises a finger grip having the reflex hammer integrally formed thereon.

In embodiments, there is provided a method of using the scissors of the type described herein, wherein the method comprises cutting tissue or a material using the scissors.

In embodiments, there is provided a method of using the scissors of the types disclosed herein, wherein the method comprises measuring an angle or length value with the scissors.

In embodiments, there is provided a method of making the scissors of the types disclosed herein, wherein the method comprises forming the first and second blade members and pivotally connecting the first and second blade members.

In embodiments, there is provided medical scissors comprising a first blade member, a second blade member movably connected to the first blade member, indicia one of arranged on and formed on the first blade member, and a projecting portion arranged on one of the first and second blade members. The projecting portion is oriented generally perpendicular to a main surface of one of the first and second blade members.

In embodiments, the surgical scissors are one of surgical scissors and bandage scissors.

In embodiments, the indicia comprises at least one of length indicia and angle indicia.

In embodiments, the indicia comprises length indicia and angle indicia.

In embodiments, there is provided a method of using the scissors of the types disclosed herein, wherein the method comprises cutting tissue or a material using the scissors and/or measuring an angle or length value with the scissors.

In embodiments, there is provided a method making the scissors of the types disclosed herein, wherein the method comprises forming the first and second blade members and pivotally connecting the first and second blade members.

In embodiments, there is provided medical shears, comprising a first blade member, a second blade member movably connected to the first blade member, a first indicia arranged adjacent a cutting edge portion of the first blade member, and a second different indicia arranged on the first blade member adjacent an edge opposite the cutting edge portion of the first blade member.

In embodiments, the scissors may further comprise a reflex hammer coupled to one of the first and second blade members.

In embodiments, the first indicia comprises angle indicia and the second indicia comprises length indicia, wherein a projecting portion is arranged on one of the first and second blade members, and wherein the projecting portion is oriented generally perpendicular to a main surface of one of the first and second blade members.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a front side view of one non-limiting embodiment of the scissors according to the invention The scissors are shown in an open configuration and show the cutting blades defining a 60 degree open position;

FIG. 2 shows a rear side view of FIG. 1;

FIG. 3 shows another front side view of FIG. 1 but with the cutting blades defining a 45 degree open position;

FIG. 4 shows another front side view of FIG. 1 but with the cutting blades defining a 30 degree open position;

FIG. 5 shows a front right side perspective view of FIG. 4;

FIG. 6 shows a front left side perspective view of FIG. 4;

FIG. 7 shows a rear side view of the scissors shown FIG. 1 but with the cutting blades just slightly opened;

FIG. 8 shows a front left side perspective view of another embodiment of the scissors. The cutting blades are shown in the substantially closed position. In this position, a user can grip the closed cutting blades portion of the scissors with one hand and use the scissors as a reflex hammer by causing the pointed thumb grip to impact a portion of a patient's body, e.g., a knee, during a reflex testing procedure;

FIG. 9 shows a front right side perspective view of FIG. 8 but with the cutting blades defining a 30 degree open position; and

FIG. 10 shows a comparison of non-limiting embodiments of the scissors in accordance with the invention with a larger scissors on the left utilizing a reflex hammer and with a smaller scissors on the right utilizing no reflex hammer.

### DETAILED DESCRIPTION OF THE INVENTION

This invention relates generally to medical, bandage or surgical scissors. The invention also relates to scissors which utilize at least one cutting blade portion that includes indicia. The invention also relates to a method of using the scissors as well as a method of making the scissors. The invention also relates to medical scissors that are ergonomic and which utilize both indicia indicating length values and angle values. Non-limiting embodiments of the invention relate to scissors which optionally utilize at least one finger grip that is shaped and/or configured for use as a reflex hammer.

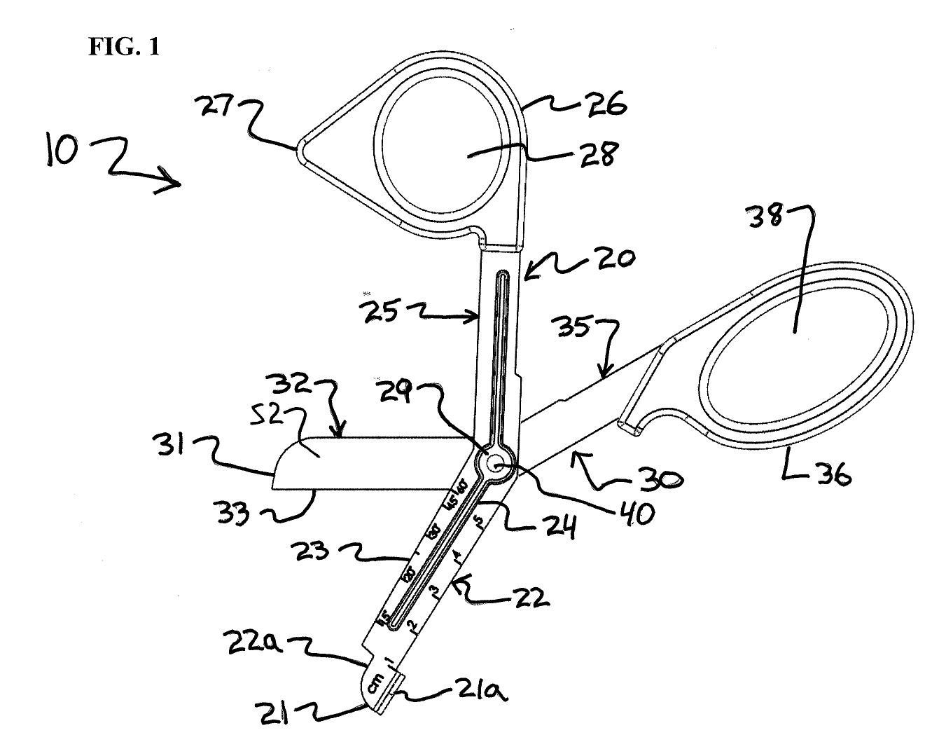

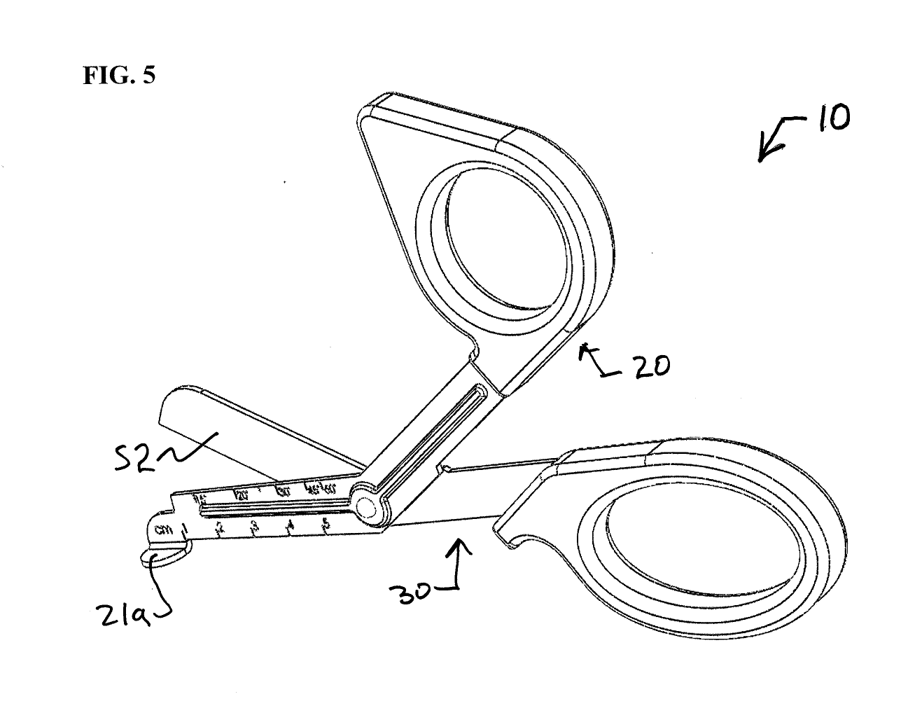

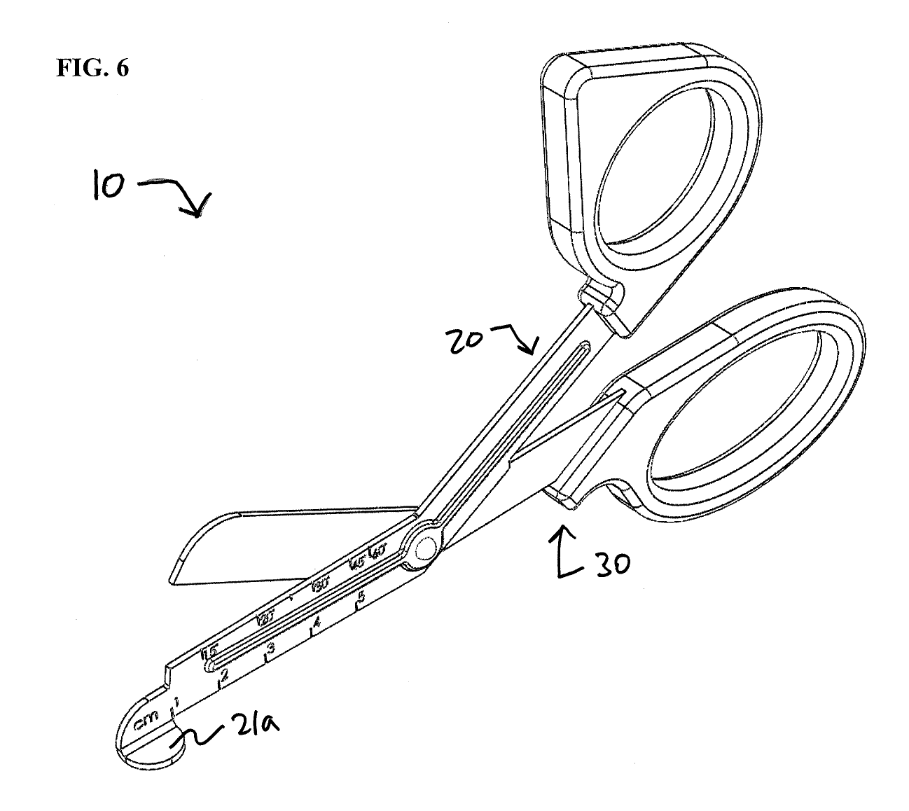

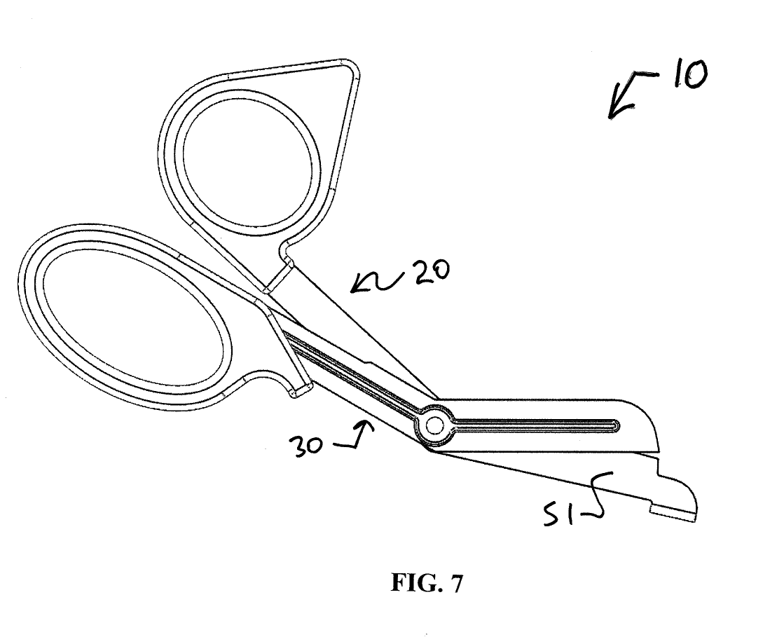

FIGS. 1-7 show a non-limiting embodiment of medical or surgical shears or scissors 10. The scissors 10 include a first blade member 20 movably, e.g., pivotally, connected to a second blade member 30. The connection 40 can be of any type such as a rivet, screw, fastener, etc. The material of the blades 20 and 30 and the connection 40 can be that which is typically used in comparable surgical or medical shears such as surgical or medical grade steel. Moreover, the blades 20 and 30 can be formed by a metal machining, metal deformation or punching, forging and/or casting. Sharpening of the cutting edge can occur in any desired way such as grinding.

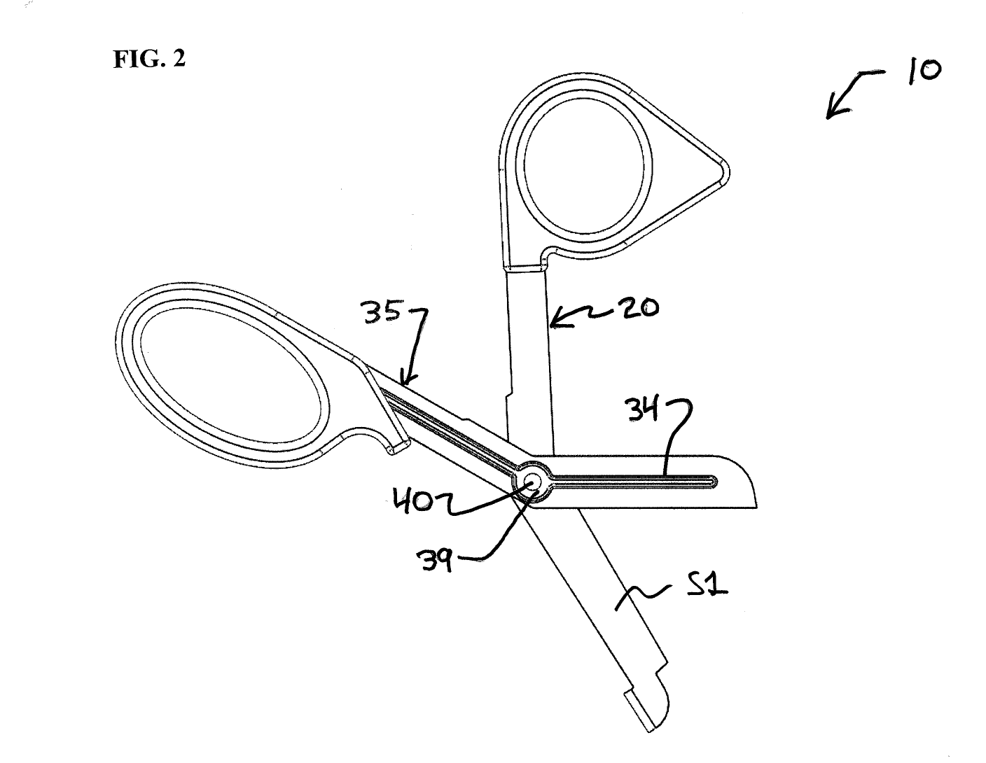

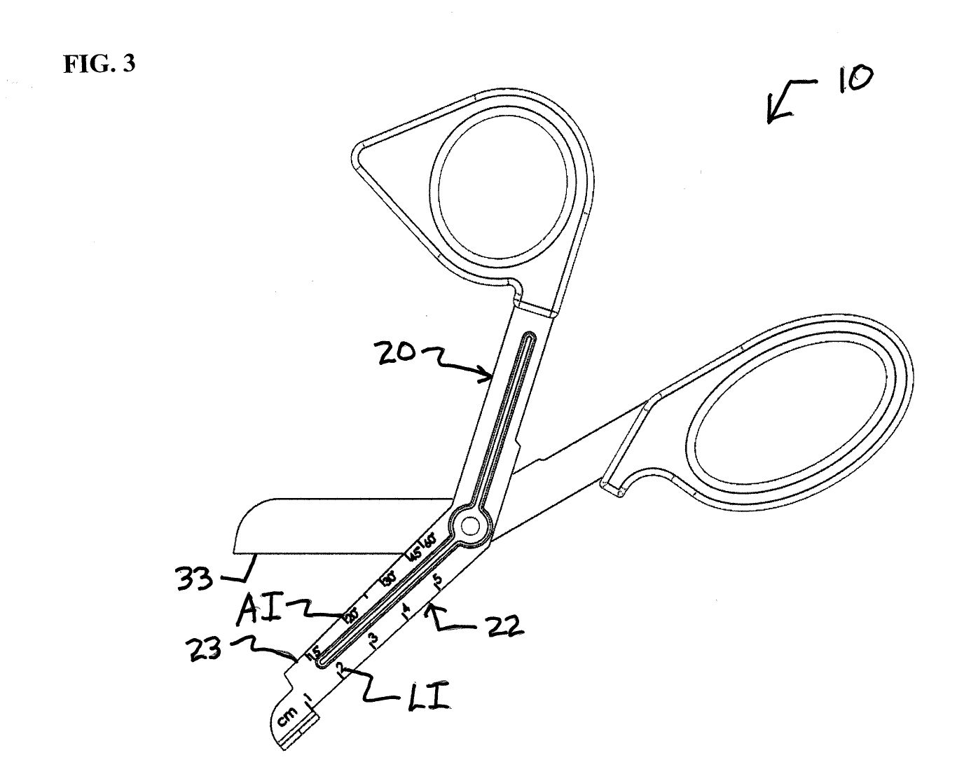

The first blade 20 includes a proximal end 21 and a cutting portion 22. A projecting portion 21a is arranged at the proximal end 21 and projects generally perpendicular to a main surface S1 (see FIG. 2), i.e., the surface facing the second blade member 30, of the first blade 20. The proximal end 21 is preferably rounded, i.e., has a blunted edge 22a, as shown in FIG. 1. A cutting edge 23 defines one edge of the blade member 20 and is generally a straight edge, although it could also be curved inwardly or outwardly. Indicia AI (see FIG. 3) is arranged adjacent the cutting edge 23 and provides information to the user regarding angle of the edge 33 in relation of the planar surface of the projection 21a. Other indicia LI (see FIG. 3) is arranged adjacent the edge opposite the cutting edge 23 and provides information to the user regarding length which allows the user to use the scissors as a scale or ruler The values for the angle can be, e.g., in whole number increments such as increments of 5 and or 10 degrees as shown in FIG. 1. The values for the length can be, e.g., in whole number increments such as increments of 1 centimeter (cm) with the width of the projection 21a being one increment or, e.g., 1 cm, as shown in FIG. 1. The indicia AI and LI can be formed in the blade 20 via etching, printing, or know ways.

The first blade 20 also includes an optional reinforcing rib 24 that can extend to a reinforced connection section 29. A lever or handle portion 25 is coupled to or preferably integrally formed with the cutting portion 22. Additionally, a finger grip 26 having a finger receiving opening 28 is arranged at a distal end of the section 25. In embodiments, the grip 26 is optionally shaped as a reflex hammer having hammer portion 27. The grip 26 can, in non-limiting embodiments, be made of a synthetic resin, e.g., medical grade resin, which is molded onto or otherwise formed and connected the distal end of the section 25.

The second blade member 30 includes a proximal end 31 and a cutting portion 32 having a main surface, i.e., the surface facing the first blade member 20. The proximal end 31 is preferably rounded, i.e., has a blunted edge, as shown in FIG. 1. A cutting edge 33 defines one edge of the blade member 30 and is generally a straight edge, although it could also be curved inwardly or outwardly. Unlike the first blade member 20, the second blade member 30 need not have any indicia. However, in embodiments, it could include indicia instead of the first blade member 20. Alternatively, it can include indicia different from that of the first blade member 20.

The second blade 30 also includes an optional reinforcing rib 34 (see FIG. 2) that can extend to a reinforced connection section 39. A lever or handle portion 35 is coupled to or preferably integrally formed with the cutting portion 32. Additionally, a finger grip 36 having a finger receiving opening 38 (e.g., that is oval in shape and generally sized for a typical user's thumb) is arranged at a distal end of the section 35. In embodiments (not shown), the grip 36 is optionally shaped as a reflex hammer having hammer portion instead of the other finger grip 26. The grip 36 can, in non-limiting embodiments, be made of a synthetic resin, e.g., medical grade resin, which is molded onto or otherwise formed and connected the distal end of the section 35.

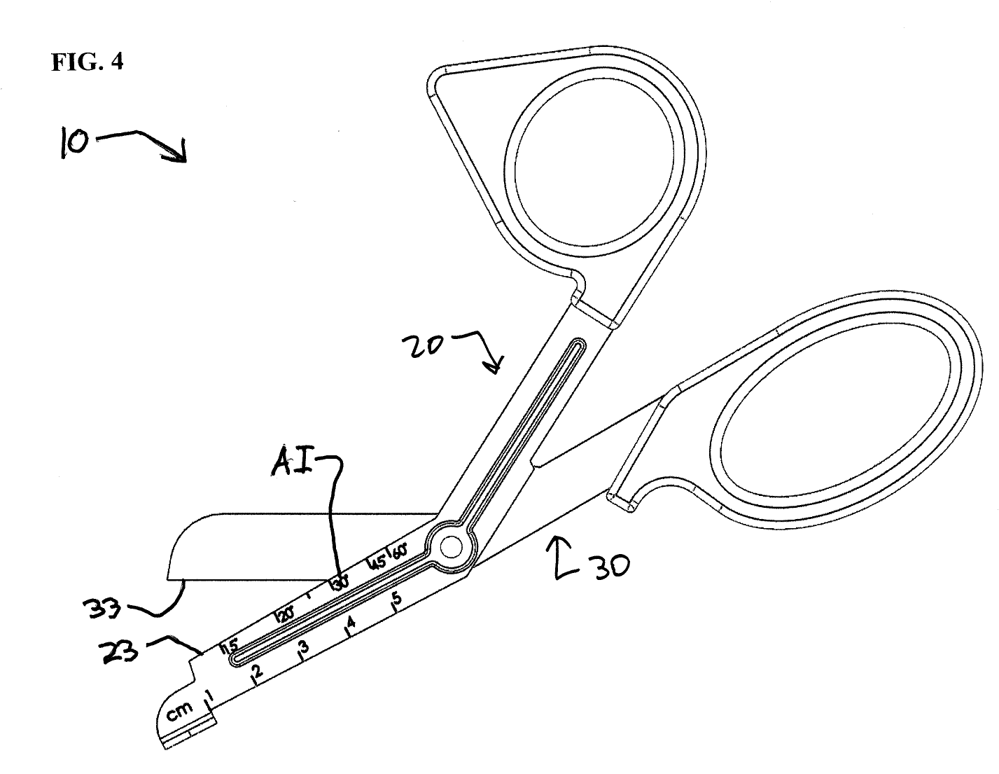

As should be apparent from FIGS. 3 and 4, cutting with the scissors 10 occurs when the first and second blade members 20 and 30 are moved so that cutting edges 23 and 33 are brought into contact and slide past each other. Where the edges 23 and 33 meet and form an intersection, one can record the angle of cutting as exemplified by the angle of 30 degrees in FIG. 4.

In embodiments, the blade 20 is a one-piece integrally formed member and the finger grip 26 is a one-piece member molded onto the one-piece blade member 20. Similarly, the blade 30 is a one-piece integrally formed member and the finger grip 36 is a one-piece member molded onto the one-piece blade member 30.

In embodiments, the connector 40 is a one-piece integrally formed member and can be press-fit into openings formed in the blade members 20 and 30, or otherwise permanently and non-removably connected to the blade members 20 and 30.

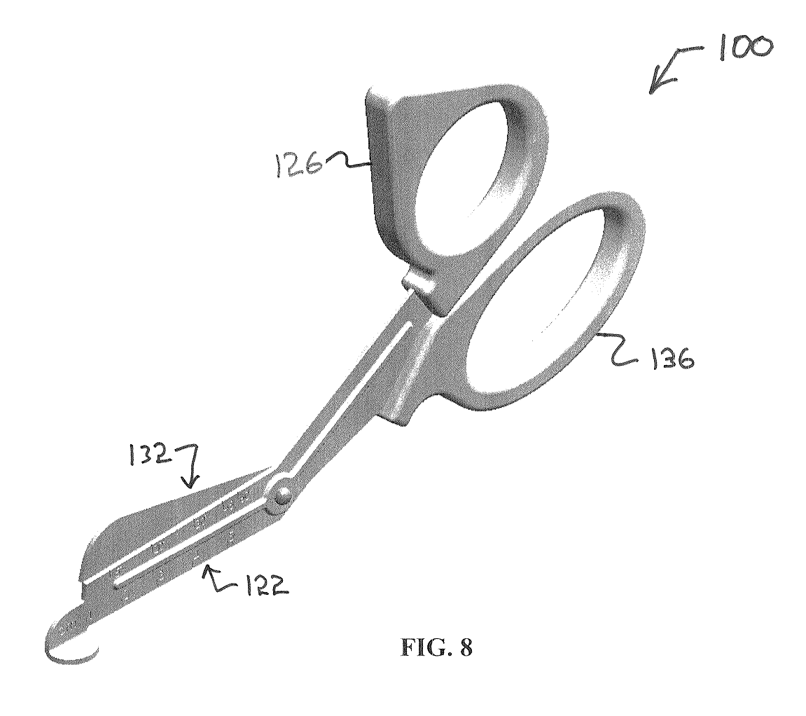

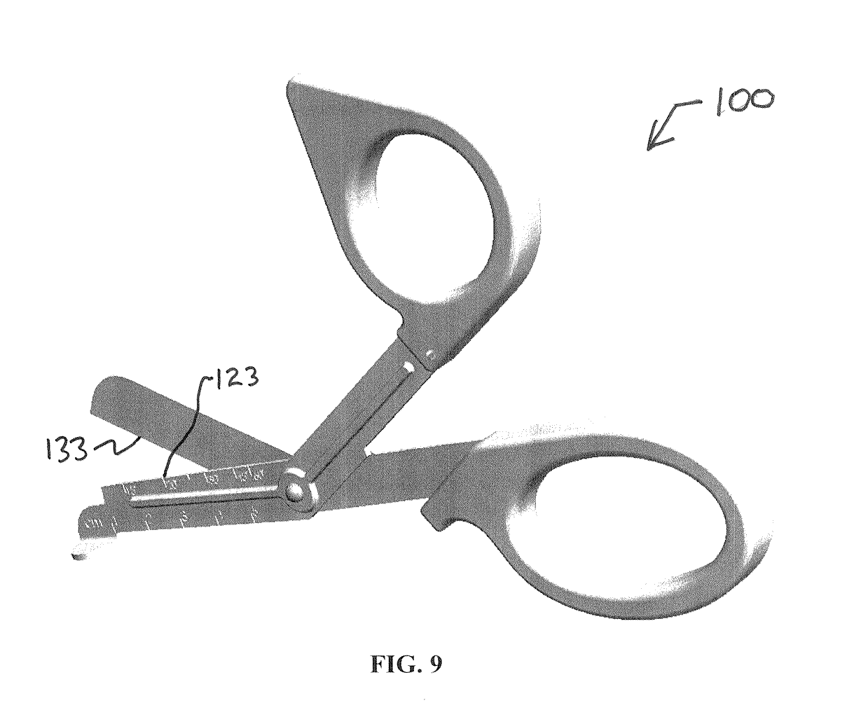

The scissor 100 shown in FIGS. 8 and 9 are similar to the scissors 10 of FIGS. 1-7 with comparable features increased by 100. In this embodiment, the blades can be formed by forging or casting, and include indicia that is formed in/on one of the blades via and/or during the forging or casting process.

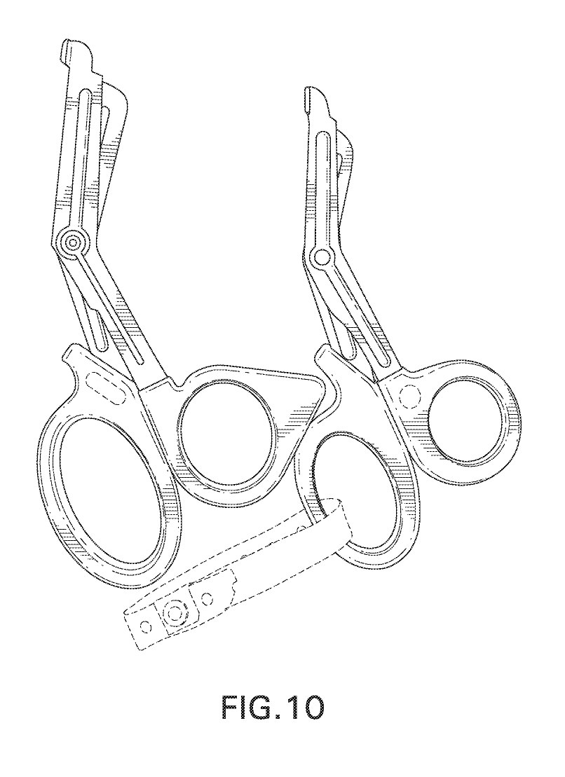

The two scissors shown in FIG. 10 offer a comparison with the scissors shown on the left being larger (approximately 20 cm or about 8 inches) and having a reflex hammer. The scissors shown on the right are smaller and lack a reflex hammer. In embodiments, the overall length of the smaller scissors shown on the right can be approximately 18.4 cm (7.25 inches). These scissors, like those shown on the left, can utilize length indicia and/or angle indicia on the cutting portion of one or more of the first and second blade members. Both smaller scissors and the larger scissor embodiments can also utilize, by way of non-limiting example, a 5 cm length cutting portion as compared to the 6 cm cutting portion utilize in the scissors shown in FIG. 1.

The scissor or shears embodiments as described herein can also utilize one or more features disclosed in the prior art documents expressly incorporated by reference herein. Furthermore, one or more of the various parts of the device such as the blade members can preferably be made as one-piece structures by shaping and bending and/or punching surgical grade steel and then forming the medical grade synthetic resin finger grips using, e.g., an over-molding process, when doing so, e.g., reduces costs of manufacture. In embodiments, the indicia have formed in the blade member during foaming, e.g., punching and/or forging, of the same. In embodiments, other known techniques for creating and/or applying indicia on medical devices can also be utilized. Non-limiting materials for most of the parts include medical grade steels and synthetic resins such as those approved for surgical scissors or other medical devices. Furthermore, the invention also contemplates that any or all disclosed features of one embodiment may be used on other disclosed embodiments, to the extent such modifications function for their intended purpose.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

### LIST OF REFERENCE NUMBERS

Scissors 10, 100;

First blade member 20, 200;

Second blade member 30, 300;

Pivot Connection member 40, 400;

Proximal end of first blade member 21, 121;

Cutting portion of first blade member 22, 122;

Proximal indentation of first blade member 22a, 122a;

Cutting edge of first blade member 23, 123;

Reinforcing rib of first blade member 24, 124;

Lever portion of first blade member 25, 125;

Finger grip of first blade member 26, 126;

Hammer portion of first blade member 27, 127;

Finger opening of first blade member 28, 128;

Pivot connecting area of first blade member 29, 129;

Proximal end of second blade member 31, 131;

Cutting portion of second blade member 32, 132;

Cutting edge of second blade member 33, 133;

Reinforcing rib of second blade member 34, 134;

Lever portion of second blade member 35, 135;

Finger grip of second blade member 36, 136;

Finger opening of second blade member 38, 138; and

Pivot connecting area of first blade member 39, 139.

What is claimed:

1. Scissors comprising:
a first blade member;
a second blade member movably connected to the first blade member; and
at least one of:
indicia arranged on at least one of the first and second blade members;
indicia formed in at least one of the first and second blade members.

2. The scissors of claim 1, further comprising a reflex hammer arranged on a handle coupled to at least one of the first and second blade members.

3. The scissors of claim 1, wherein the scissors are one of surgical scissors, medical scissors and bandage scissors.

4. The scissors of claim 1, wherein the indicia is arranged on a first blade member and a reflex hammer is coupled to the first blade member.

5. The scissors of claim 1, wherein the indicia comprises at least one of length indicia and angle indicia.

6. The scissors of claim 1, wherein the indicia comprises length indicia and angle indicia.

7. The scissors of claim 1, wherein the indicia is arranged on the first blade member adjacent a cutting portion of the first blade member and a lever portion of the first blade member comprises a finger grip having the reflex hammer integrally formed thereon.

8. A method of using the scissors of claim 1, the method comprising:
cutting tissue or a material using the scissors.

9. A method of using the scissors of claim 1, the method comprising:
measuring an angle or length value with the scissors.

10. A method of making the scissors of claim 1, the method comprising:
forming the first and second blade members; and
pivotally connecting the first and second blade members.

11. Medical scissors comprising:
a first blade member;
a second blade member movably connected to the first blade member;
indicia one of arranged on and formed on a cutting portion of the first blade member; and
a projecting portion arranged on one of the first and second blade members,
wherein the projecting portion is oriented generally perpendicular to a main surface of one of the first and second blade members.

12. The scissors of claim 11, wherein the surgical scissors are one of surgical scissors and bandage scissors.

13. The scissors of claim 11, wherein the indicia comprises at least one of length indicia and angle indicia.

14. The scissors of claim 11, wherein the indicia comprises length indicia and angle indicia.

15. A method of using the scissors of claim 11, the method comprising:
cutting tissue or a material using the scissors.

16. A method of using the scissors of claim 11, the method comprising:
measuring an angle or length value with the scissors.

17. A method of making the scissors of claim 11, the method comprising:
forming the first and second blade members; and
pivotally connecting the first and second blade members.

18. Medical shears, comprising:
a first blade member;
a second blade member movably connected to the first blade member;
a first indicia arranged adjacent a cutting edge portion of the first blade member; and
a second different indicia arranged on the first blade member adjacent an edge opposite the cutting edge portion of the first blade member.

19. The scissors of claim 18, further a reflex hammer coupled to one of the first and second blade members.

20. The scissors of claim 18, wherein the first indicia comprises angle indicia and the second indicia comprises length indicia, wherein a projecting portion is arranged on one of the first and second blade members, and wherein the projecting portion is oriented generally perpendicular to a main surface of one of the first and second blade members.
